(19) United States
(12) Patent Application Publication
(10) Pub. No.: US 2005/0239846 A1
(43) Pub. Date: Oct. 27, 2005

(54) Use of vanilloid receptor antagonists for the treatment of pain

(76) Inventors: John Beresford Davis, Harlow (GB); Wendy Joyce Winchester, Harlow (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/527,481

(22) PCT Filed: Sep. 10, 2003
(86) PCT No.: PCT/EP03/10261

§ 371 Date: Mar. 11, 2005

(30) Foreign Application Priority Data
Sep. 12, 2002 (GB) 0221157.1

(51) Int. Cl. A61K 31/4745; A61K 35/78

(52) U.S. Cl. 514/343; 424/760

(57) ABSTRACT

A method for the treatment and/or prophylaxis of pelvic pain, renal colic, biliary colic, functional dyspepsia, Barrett's metaplasia, dysphagia and pain associated therewith, in humans or non-human mammals, which method comprises the administration of an effective, non-toxic and pharmaceutically acceptable amount of a vanilloid receptor antagonist.

This invention relates to a novel treatment and in particular to a method for the treatment and/or prophylaxis of pelvic pain, renal colic, biliary colic, functional dyspepsia, Barrett's metaplasia, dysphagia and pain associated therewith.

Vanilloids are a class of natural and synthetic compounds that are characterised by the presence of a vanillyl (4-hydroxy 3-methoxybenzyl) group or a functionally equivalent group. Vanilloid Receptor (VR-1), whose function is modulated by such compounds, has been widely studied and is extensively reviewed by Szallasi and Blumberg (The American Society for Pharmacology and Experimental Therapeutics, 1999, Vol. 51, No. 2.).

A wide variety of vanilloid compounds of different structures are known in the art, for example those disclosed in European Patent Application Numbers, EP 0 347 000 and EP 0 401 903, UK Patent Application Number GB 2226313 and International Patent Application, Publication Number WO 92/09285. Particularly notable examples of vanilloid compounds or vanilloid receptor modulators are capsaicin or trans 8-methyl-N-vanillyl-6-nonenamide which is isolated from the pepper plant, capsazepine (Tetrahedron, 53, 1997, 4791) and olvanil or —N-(4-hydroxy-3-methoxybenzyl)oleamide (J. Med. Chem., 36, 1993, 2595).

U.S. Pat. No. 3,424,760 and U.S. Pat. No. 3,424,761 both describe a series of 3-Ureidopyrrolidines that are said to exhibit analgesic, central nervous system, and pyschopharmacological activities.

International Patent Application, Publication Number WO 01/021577 discloses the preparation of a series of N-tetrahydronaphthalenyl derivatives, having biological activity as melanin-concentrating hormone antagonists.

International Patent Application, Publication Number WO 02/08221 discloses diaryl piperazine and related compounds which bind with high selectivity and high affinity to vanilloid receptors, especially Type I vanilloid receptors, also known as capsaicin or VR-1 receptors. The compounds are said to be useful in the treatment of chronic and acute pain conditions, itch and urinary incontinence. International Patent Application, Publication Numbers WO 02/16317, WO 02/16318 and WO 02/16319 suggest that compounds having a high affinity for the vanilloid receptor are useful for treating stomach-duodenal ulcers.

International Patent Applications, Publication Numbers WO 02/072536, WO 02/090326, WO 03/022809 and WO 03/053945; and International Patent Application Number PCT/GB03/00608 also describe a variety of compounds having activity as vanilloid receptor antagonists.

It is now surprisingly indicated that compounds having activity as vanilloid receptor antagonists have activity in the treatment and/or prophylaxis of pelvic pain, renal colic, biliary colic, functional dyspepsia, Barrett's metaplasia, dysphagia and pain associated therewith.

Accordingly, the invention provides a method for the treatment and/or prophylaxis of pelvic pain, renal colic, biliary colic, functional dyspepsia, Barrett's metaplasia, dysphagia and pain associated therewith, in humans or non-human mammals, which method comprises the administration of an effective, non-toxic and pharmaceutically acceptable amount of a vanilloid receptor antagonist.

Suitably, the invention provides a method for the treatment and/or prophylaxis of pelvic pain.

Suitably, the invention provides a method for the treatment and/or prophylaxis of renal colic or pain associated therewith.

Suitably, the invention provides a method for the treatment and/or prophylaxis of biliary colic or pain associated therewith.

Suitably, the invention provides a method for the treatment and/or prophylaxis of functional dyspepsia or pain associated therewith, such as, heartburn.

Suitably, the invention provides a method for the treatment and/or prophylaxis of Barrett's metaplasia or pain associated therewith.

Suitably, the invention provides a method for the treatment and/or prophylaxis of dysphagia or pain associated therewith.

Suitably, the vanilloid receptor antagonist is an antagonist of the vanilloid receptor-1.

Suitable vanilloid receptor antagonists for use in accordance with the present invention include those disclosed in European Patent numbers EP 0 347 000 and EP 0 401 903; UK Patent Application Number GB 2226313; International Patent Applications, Publication Numbers WO 92/09285, WO 01/021577, WO 02/08221, WO 02/16317, WO 02/16318, WO 02/16319, WO 02/072536, WO 02/090326, WO 03/022809 and WO 03/053945; International Patent Application Number PCT/GB03/00608; and U.S. Pat. No. 3,424,760 and U.S. Pat. No. 3,424,761.

Preferred vanilloid receptor antagonists for use in accordance with the present invention include those disclosed in International Patent Applications, Publication Numbers WO 02/072536, WO 02/090326, WO 03/022809 and WO 03/053945; and International Patent Application Number PCT/GB03/00608.

A particularly preferred vanilloid receptor antagonist for use in accordance with the present invention is N-(2-Bromophenyl)-N′-[((R)-1-(5-trifluoromethyl-2-pyridyl)pyrrolidin-3-yl)]urea (hereafter referred to as “Compound A”), or a pharmaceutically acceptable derivative thereof. Compound A is disclosed at Example 1 of International Patent Application, Publication Number WO 03/022809.

All publications, including but not limited to patents and patent applications, cited in this specification are herein incorporated by reference as if each individual publication were specifically and individually indicated to be incorporated by reference herein as though fully set forth.

Certain vanilloid receptor antagonists may exist in one of several tautomeric forms, all of which are encompassed by the present invention as individual tautomeric forms or as mixtures thereof. Where a vanilloid receptor antagonist contains a chiral carbon, and hence exists in one or more stereoisomeric forms or where one or more geometric isomers exist, it will be appreciated that the method of the present invention encompasses all of the said forms of the vanilloid receptor antagonists whether as individual isomers or as mixtures of isomers, including racemates.

When used herein the term ‘vanilloid receptor antagonist’ relates to an antagonist, such as a small molecular weight antagonist, of the vanilloid receptor. It will be appreciated that the term also embraces suitable pharmaceutically acceptable derivatives thereof.

Vanilloid receptor antagonist activity may be assessed by use of the methodologies disclosed in the above-mentioned patent applications, such as, WO 02/08221, WO 02/16317 and WO 02/090326.

Suitable pharmaceutically acceptable derivatives of a vanilloid receptor antagonist are, for example, salts and solvates.

Suitable pharmaceutically acceptable derivatives of any particular vanilloid receptor antagonist include those disclosed in the above-mentioned publications.

Suitable pharmaceutically acceptable salts include salts derived from appropriate acids, such as acid addition salts, or bases.

Suitable pharmaceutically acceptable salts include metal salts, such as for example aluminium, alkali metal salts such as lithium, sodium or potassium, alkaline earth metal salts such as calcium or magnesium and ammonium or substituted ammonium salts, for example those with lower alkylamines such as triethylamine, hydroxy alkylamines such as 2-hydroxyethylamine, bis-(2-hydroxyethyl)-amine or tri-(2-hydroxyethyl)-amine, cycloalkylamines such as bicyclohexylamine, or with procaine, dibenzylpiperidine, N-benzyl-b-phenethylamine, dehydroabietylamine, N,N′-bisdehydroabietylamine, glucamine, N-methylglucamine or bases of the pyridine type such as pyridine, collidine, quinine or quinoline.

Suitable acid addition salts include pharmaceutically acceptable inorganic salts such as the sulfate, nitrate, phosphate, borate, hydrochloride and hydrobromide and pharmaceutically acceptable organic acid addition salts such as acetate, tartrate, maleate, citrate, succinate, benzoate, ascorbate, methane-sulfonate, α-keto glutarate and α-glycerophosphate, especially the maleate salt.

The vanilloid receptor antagonists referred to herein are conveniently prepared according to the methods disclosed in the above mentioned patent publications in which they are disclosed.

The salts and/or solvates of the vanilloid receptor antagonists referred to herein may be prepared and isolated according to conventional procedures for example those disclosed in the above mentioned patent publications.

The present invention also provides a vanilloid receptor antagonist or a pharmaceutically acceptable derivative thereof, for use in the treatment and/or prophylaxis of pelvic pain, renal colic, biliary colic, functional dyspepsia, Barrett's metaplasia, dysphagia and pain associated therewith.

The present invention also provides a vanilloid receptor antagonist or a pharmaceutically acceptable derivative thereof, for use in the manufacture of a medicament for the treatment and/or prophylaxis of pelvic pain, renal colic, biliary colic, functional dyspepsia, Barrett's metaplasia, dysphagia and pain associated therewith.

In the above-mentioned method the vanilloid receptor antagonist, may be administered per se or, preferably, as a pharmaceutical composition also comprising a pharmaceutically acceptable carrier.

In the treatment of the invention, the vanilloid receptor antagonist mentioned herein is formulated and administered in accordance with the methods disclosed in the above mentioned patent applications and patents.

Accordingly, the present invention also provides a pharmaceutical composition for the treatment and/or prophylaxis of pelvic pain, renal colic, biliary colic, functional dyspepsia, Barrett's metaplasia, dysphagia and pain associated therewith, which composition comprises a vanilloid antagonist, or a pharmaceutically acceptable derivative thereof, and a pharmaceutically acceptable carrier therefor.

As used herein the term ‘pharmaceutically acceptable’ embraces compounds, compositions and ingredients for both human and veterinary use: for example the term ‘pharmaceutically acceptable salt’ embraces a veterinarily acceptable salt.

The composition may, if desired, be in the form of a pack accompanied by written or printed instructions for use.

Usually the pharmaceutical compositions of the present invention will be adapted for oral administration, although compositions for administration by other routes, such as by injection and percutaneous absorption are also envisaged.

Particularly suitable compositions for oral administration are unit dosage forms such as tablets and capsules. Other fixed unit dosage forms, such as powders presented in sachets, may also be used.

In accordance with conventional pharmaceutical practice the carrier may comprise a diluent, filler, disintegrant, wetting agent, lubricant, colourant, flavourant or other conventional adjuvant.

Typical carriers include, for example, microcrystalline cellulose, starch, sodium starch glycollate, polyvinylpyrrolidone, polyvinylpolypyrrolidone, magnesium stearate, sodium lauryl sulphate or sucrose.

Suitable dosages of the vanilloid receptor antagonist include the known doses for these compounds as described or referred to in reference texts such as the British and US Pharmacopoeias, Remington's Pharmaceutical Sciences (Mack Publishing Co.), Martindale The Extra Pharmacopoeia (London, The Pharmaceutical Press) (for example see the 31st Edition page 341 and pages cited therein) or the above mentioned publications or doses which can be determined by standard procedures.

The solid oral compositions may be prepared by conventional methods of blending, filling or tabletting. Repeated blending operations may be used to distribute the active agent throughout those compositions employing large quantities of fillers. Such operations are of course conventional in the art. The tablets may be coated according to methods well known in normal pharmaceutical practice, in particular with an enteric coating.

Oral liquid preparations may be in the form of, for example, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel, hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl p-hydroxybenzoate or sorbic acid; and if desired conventional flavouring or colouring agents.

For parenteral administration, fluid unit dosage forms are prepared utilizing the compound and a sterile vehicle, and, depending on the concentration used, can be either suspended or dissolved in the vehicle. In preparing solutions the compound can be dissolved in water for injection and filter sterilized before filling into a suitable vial or ampoule and sealing. Advantageously, adjuvants such as a local anaesthetic, a preservative and buffering agents can be dissolved in the vehicle. To enhance the stability, the composition can be frozen after filling into the vial and the water removed under vacuum. Parenteral suspensions are prepared in substantially the same manner, except that the compound is suspended in the vehicle instead of being dissolved, and sterilization cannot be accomplished by filtration. The compound can be sterilized by exposure to ethylene oxide before suspending in the sterile vehicle. Advantageously, a surfactant or wetting agent is included in the composition to facilitate uniform distribution of the compound.

Compositions may contain from 0.1% to 99% by weight, preferably from 10-60% by weight, of the active material, depending upon the method of administration.

Compositions may, if desired, be in the form of a pack accompanied by written or printed instructions for use.

The compositions are formulated according to conventional methods, such as those disclosed in standard reference texts, for example the British and US Pharmacopoeias, Remington's Pharmaceutical Sciences (Mack Publishing Co.), Martindale The Extra Pharmacopoeia (London, The Pharmaceutical Press) and Harry's Cosmeticology (Leonard Hill Books).

No adverse toxicological effects are expected for the compositions or methods of the invention in the above mentioned dosage ranges.

1. A method for the treatment and/or prophylaxis of pelvic pain, renal colic, biliary colic, functional dyspepsia, Barrett's metaplasia, dysphagia and pain associated therewith, in humans or non-human mammals, which method comprises the administration of an effective, non-toxic and pharmaceutically acceptable amount of a vanilloid receptor antagonist.

2. A method according to claim 1, for the treatment and/or prophylaxis of renal colic or pain associated therewith.

3. A method according to claim 1, for the treatment and/or prophylaxis of biliary colic or pain associated therewith.

4. A method according to claim 1, for the treatment and/or prophylaxis of functional dyspepsia or pain associated therewith, such as, heartburn.

5. A method according to claim 1, for the treatment and/or prophylaxis of Barrett's metaplasia or pain associated therewith.

6. A method according to claim 1, for the treatment and/or prophylaxis of dysphagia or pain associated therewith.

7. A method according to claim 1, wherein the vanilloid receptor antagonist is an antagonist of the vanilloid receptor-1.

8. A method according to claim 7, wherein the vanilloid receptor-1 antagonist is N-(2-Bromophenyl)-N′-[((R)-1-(5-trifluoromethyl-2-pyridyl)pyrrolidin-3-yl)]urea.

9. A pharmaceutical composition for the treatment and/or prophylaxis of pelvic pain, renal colic, biliary colic, functional dyspepsia, Barrett's metaplasia, dysphagia and pain associated therewith, which composition comprises a vanilloid antagonist, or a pharmaceutically acceptable derivative thereof, and a pharmaceutically acceptable carrier therefor.

10. (canceled)

11. (canceled)
